(19) United States
(12) Patent Application Publication
(10) Pub. No.: US 2022/0288125 A1
(43) Pub. Date: Sep. 15, 2022

(54) Nutritional Composition for Improving Sleep

(71) Applicant: Stokely-Van Camp, Inc., Chicago, IL (US)

(72) Inventor: Lara Nyman, North Haven, CT (US)

(21) Appl. No.: 17/684,861

(22) Filed: Mar. 2, 2022

Related U.S. Application Data
(60) Provisional application No. 63/161,168, filed on Mar. 15, 2021.

(51) Int. Cl. A61K 35/20; A61K 31/405; A61K 31/4172; A61K 31/198; A61K 31/718; A61K 31/7076; A61K 9/00; A61P 25/20

(52) U.S. Cl. CPC A61K 35/20; A61K 31/405; A61K 31/4172; A61K 31/198; A61K 31/718; A61K 31/7076; A61K 9/0095; A61P 25/20

(57) ABSTRACT

A nutritional composition that includes from about 75% to about 95% of a first protein source that includes from about 85% to about 99% of protein by weight of the first protein source to provide an endogenous source of tryptophan in an amount between about 1% to about 6% of tryptophan by weight of the first protein source; and from about 0.5% to about 2% of a second protein source by weight of the nutritional composition, the second protein source consisting of an exogenous source of tryptophan such that the nutritional composition provides a ratio of tryptophan to large neutral amino acids in the range from about 0.080:1 to about 0.167:1.

This application claims priority to U.S. Application No. 63/161,168 filed Mar. 15, 2021, the entire contents of which are incorporated herein by reference.

Nutritional compositions that may be useful for addressing qualitative and quantitative aspects of sleep such as, but not limited to, reducing sleep onset latency, i.e., the amount of time needed to fall asleep, are described. Methods of supporting or enhancing at least one and, in some instances, at least two or more, qualitative and/or quantitative aspect(s) of sleep in a subject include administering the described compositions to a subject, where the aspects may be selected from (i) promoting falling asleep; (ii) inducing or supporting a healthy sleep pattern; (iii) increasing the total sleep time, (iv) improving sleep efficiency, (v) reducing or preventing sleep disturbances/sleeping more time while in bed; (vi) increasing subjective and/or objective measures of deep sleep; (vii) feeling more refreshed at waking up; (viii) feeling more energized and/or having a better mood while awake after sleeping.

### BACKGROUND

Disturbed sleep may negatively influence physical health, cognitive performance, metabolism, and general wellbeing. Nutritional interventions represent a potential non-pharmacological means to increase sleep quality and quantity. To that end, nutritional compositions that reduce sleep onset latency and that are able to address the qualitative and quantitative aspects noted above, in a subject without adversely impairing cognitive abilities after sleep are desired.

### SUMMARY

In view of the above, the following describes a nutritional composition that includes from about 75% to about 95% of a first protein source that contains from about 60% to about 99% of protein (which may contain alpha-lactalbumin) by weight of the first protein source to provide an endogenous source of tryptophan in an amount between about 1% to about 6% of tryptophan by weight of the first protein source; and from about 0.5% to about 2% of a second protein source by weight of the nutritional composition, the second protein source consisting of an exogenous source of tryptophan such that the nutritional composition provides a ratio of tryptophan to large neutral amino acids in the range from about 0.080:1 to about 0.167:1 (the skilled artisan would understand that such ratio could likewise be expressed as a ratio of large neutral amino acids to tryptophan in the range from about 6:1 to about 12.5:1).

All references to tryptophan are meant to refer to L-tryptophan unless specifically stated otherwise. The large neutral amino acids (LNAA) may be selected from the group consisting of histidine, isoleucine, leucine, methionine, threonine, phenylalanine, valine, tyrosine, and combinations thereof. The skilled artisan will appreciate that the LNAA will typically be present in and provided by the first protein source, e.g., whey protein, whey protein concentrate, whey protein isolate, and mixtures thereof. While tryptophan is typically considered a LNAA, when calculating the ratio of tryptophan:LNAA, the amount of tryptophan present in the first protein source is not included with (considered in the calculation) or in the total amount of the LNAA.

As used in the following description, the term “endogenous tryptophan” refers to the tryptophan that is present in and is a component of the first protein source, for example, by being present in alpha-lactalbumin or present in the source of lactalbumin such as from whey protein, whey protein concentrate, whey protein isolate and mixtures thereof. Put another way endogenous tryptophan is present in the nutritional composition by virtue of the presence of the first protein and is not, itself isolated but rather is an inseparable component of the first protein. As such, and as noted above, the amount of tryptophan present in the first protein source is from about 1% to about 6%.

In contrast, the term “exogenous source of tryptophan” refers to an amount tryptophan provided by a second protein different from the first protein and that is not present in the first protein source. Accordingly, it will be understood that the “exogenous tryptophan” will provide, in the nutritional composition, an enhanced or supplemental amount of tryptophan which is greater than the theoretical amount that could be provided by the first protein source. In some instances, the exogenous source of tryptophan can be provided as 100% tryptophan. (i.e., as an isolated form of tryptophan).

According to one aspect, the first protein source may be selected from whey protein, whey protein concentrate, whey protein isolate, and mixtures thereof. According to one embodiment, the first protein source may be a whey protein concentrate that comprises between about 60% to about 85% protein by weight of the whey protein concentrate. In other aspects, the first protein source may be a whey protein isolate that comprises between about 85% to about 99% protein by weight of the whey protein isolate.

In some embodiments, the nutritional composition may include other beneficial ingredients including a carbohydrate, an amino acid other than a LNAA such as theanine, and a purine ribonucleoside monophosphate. To this end, in some embodiments, the nutritional composition does not contain and is free of tart cherry juice, valerian, or both tart cherry juice and valerian.

In one aspect, the carbohydrate may be present in the nutritional composition in an amount from about 3% to about 10% and in some embodiments the carbohydrate may be a high glycemic index carbohydrate. In this regard, a high glycemic index carbohydrate refers to a carbohydrate that has a glycemic index between about 85 and about 105. The high glycemic carbohydrate may be provided from any suitable food grade source. In that regard, the term “food grade” refers to materials that are safe for human consumption. It has been found that a suitable high glycemic index carbohydrate may include one or more maltodextrins. In one aspect, the high glycemic index carbohydrate is a maltodextrin that has a dextrose equivalent (DE) from about 3 to about 20 or about 15 to about 19, or about 18.

In one aspect, the amino acid other than the LNAA may be theanine which may be present in the nutritional composition in amounts from about 0.25% to about 4% or from about 0.25% to about 2% by weight of the nutritional composition.

In one aspect, the purine ribonucleoside monophosphate may include adenosine-5′-monophosphate in an amount of about 0.00003% to about 0.0004% or from about 0.00005% to about 0.0002% by weight of the nutritional composition.

The described nutritional composition may be provided in any suitable form for oral ingestion by a subject. In that regard, the nutritional composition may be provided as a powder, tablet, capsule or as a liquid including a beverage.

Methods of using the described composition are also contemplated. In some aspects, the method includes supporting or enhancing at least one, and in some instances, at least two or more, qualitative and/or quantitative aspect(s) of sleep in a subject. The qualitative and/or quantitative aspects may include (i) promoting falling asleep; (ii) inducing or supporting a healthy sleep pattern; (iii) increasing total sleep time, (iv) improving sleep efficiency, (v) reducing or preventing sleep disturbances/sleeping more time while in bed; (vi) increasing subjective and/or objective measures of deep sleep; (vii) feeling more refreshed at waking up; (viii) feeling more energized and/or having a better mood during daytime comprising administering to a subject (e.g., a human) the described composition.

All percentages used or recited in this description refer to a percent by weight, unless specifically stated otherwise. Other aspects and advantages of this invention will be appreciated from the following detailed description.

### DETAILED DESCRIPTION

A nutritional composition is described that includes from about 75% to about 95% of a first protein source that includes from about 85% to about 99% of protein by weight of the first protein source and an endogenous source of tryptophan in an amount between about 1% to about 6% of tryptophan by weight of the first protein source; and from about 0.5% to about 4% of a second protein source consisting of an exogenous source of tryptophan such that the nutritional composition provides a ratio of tryptophan to large neutral amino acids (LNAA) in the range of about 0.080:1 to about 0.167:1 (the skilled artisan would appreciate that these ratios could be expressed as a ratio of LNAA to tryptophan in the range from about 6:1 to about 12.5:1). The large neutral amino acids (LNAA) may be selected from the group consisting of histidine, isoleucine, leucine, methionine, threonine, phenylalanine, valine, tyrosine and combinations thereof.

It will be appreciated by one of skill in the art that the first protein source is not capable of providing a sufficient amount of tryptophan to achieve the desired ratio of tryptophan to LNAA. Accordingly, to achieve the desired ratio of tryptophan to LNAA in the nutritional composition, the first protein source must be supplemented with tryptophan and in some instances supplemented with an exogenous source of tryptophan. In some instances, the nutritional composition contains a total amount of tryptophan in the range from about 0.5% to about 10%, or about 1% to about 10%, or about 1.5% to about 10% tryptophan, or about 1.6% to about 5%, or about 1.7% to about 4%, or about 1.8% to about 3%.

Tryptophan has been linked to sleep and alertness/attention. It is a precursor of the neurotransmitter serotonin. Active uptake of tryptophan (Trp) in the brain is influenced by its plasma concentration (and thus diet concentration), and the concentration of the competing large neutral amino acids (LNAA), which include histidine, isoleucine, leucine, methionine, threonine, phenylalanine, valine, and tyrosine. Thus, the described nutritional composition seeks to provide a beneficial ratio of tryptophan to these LNAA. To this end, the nutritional composition provides a ratio of tryptophan (Trp) to LNAA from about 0.080:1 to about 0.167:1, or from about 0.100:1 to about 0.154:1, or from about 0.111:1 to about 0.143:1, or about 0.120:1 to about 0.130:1, or about 0.125:1 to about 0.127:1 (it will be appreciated by the skilled artisan that such ratios likewise provide a ratio of LNAA to tryptophan from about 6:1 to about 12.5:1, or from about 6.5:1 to about 10:1, or about 7:1 to about 9:1, or about 7.7:1 to about 8.3:1 or about 7.9:1 to about 8.1:1).

To deliver a sufficient amount of “sleep-promoting” tryptophan, the use of a first protein source that is relatively rich in tryptophan is advantageously used. In some embodiments, the first protein source may include alpha-lactalbumin which contains a significant amount of endogenous tryptophan. While the source of tryptophan is not particularly important, in certain aspects, the first protein source is selected from whey protein, whey protein concentrate, whey protein isolate, and mixtures thereof, each of which provides a significant amount of tryptophan. Whey proteins contain, of all milk proteins, the highest levels of α-lactalbumin and, thus, tryptophan.

In one embodiment, the first protein source in the nutritional composition comprises, consists essentially of, or consists of a whey protein, a whey protein isolate (WPI), or a whey protein concentrate (WPC), and mixtures thereof. In one aspect, the first protein source is a whey protein concentrate, a whey protein isolate, or a mixture of a whey protein concentrate and a whey protein isolate. The first protein source has a protein content of at least about 50%, about 60%, about 70%, about 80%, about 85%, about 90%, about 95%, or about 98% or, in some instances between about 85% to about 99%, or between about 86% to about 98%, or between about 87% to about 97%, or between about 88% to about 96%, or between about 89% to about 95%. The whey protein, whey protein isolate, or whey protein concentrate may be derived from acid whey, cheese whey, milk microfiltration permeate and combinations thereof.

As noted above, the first protein source is present from about 75% to about 95% by weight of the nutritional composition or from about 85% to about 93% or from about 88% to about 91% or about 90%, by weight of the nutritional composition.

To achieve the desired ratio of tryptophan to LNAA, the nutritional composition includes a second protein source which provides an exogenous source of tryptophan. To this end, the second protein consists only of tryptophan, and in some instances consists of isolated tryptophan.

The second protein source is present in the nutritional composition in an amount from about 0.5% to about 4%, or from about 1% to about 3.5% or from about 2% to about 3.25% It will be understood that the second protein is present in the nutritional composition in an amount to provide a ratio of tryptophan:LNAA in the nutritional composition in a range from about 0.080:1 to about 0.167:1 and, that in the absence of the second protein source, the described ratio of tryptophan:LNAA would not be achieved.

In some embodiments, the nutritional composition may include other beneficial ingredients including a carbohydrate, an amino acid other than a LNAA, and a purine ribonucleoside monophosphate.

Therefore, in one aspect, the carbohydrate may be present in the nutritional composition in an amount from about 3% to about 10% by weight of the nutritional composition, or about 4% to about 9%, or about 5% to about 8%, or about 5.5% to about 7%, or about 6% or about 8%. In some embodiments, the carbohydrate may be a high glycemic index carbohydrate. In this regard, a high glycemic index carbohydrate refers to a carbohydrate that has a glycemic index between about 85 and about 105. The high glycemic carbohydrate may be provided from any suitable food grade source. In that regard, the term “food grade” refers to materials that are safe for human consumption. It has been found that a suitable high glycemic index carbohydrate includes one or more maltodextrins. In one aspect, the high glycemic index carbohydrate is a maltodextrin that has a dextrose equivalent (DE) from about 3 to about 20 or about 15 to about 19, or about 18.

In one aspect, the amino acid other than a LNAA may be theanine, which may be present in the nutritional composition in amounts from about 0.25% to about 4% or from about 0.25% to about 2% by weight of the nutritional composition, or about 0.5% to about 2%, or about 1% to about 2%, about 2%, or about 1.5%, or about 2.5%.

In one aspect, the purine ribonucleoside monophosphate may include adenosine-5′-monophosphate in an amount of about 0.00005% to about 0.0004%, or about 0.00005% to about 0.0003%, or about 0.00015% to about 0.00025%, or about 0.0002% by weight of the nutritional composition, or about 0.0001%.

The nutritional composition may be formulated in any desirable and suitable form for oral ingestion by a subject such as a human. For example, the nutritional composition may be in a dry, liquid or semi-liquid state. In one embodiment, unit dosage forms of the compositions may be provided for oral consumption such as, but not limited to, tablets, capsules, pills, lozenges, wafers, powders, liquids, emulsions, suspensions, solutions and the like. The nutritional composition may also have the form of or may be included in a food or a beverage, such as a dietary supplement bar or shake.

To that end, it is contemplated that the nutritional composition may contain a trivial amount of a sweetener or flavor, i.e., each present in an amount of less than 1%

In some embodiments, the nutritional composition may be provided as a liquid suspension or as a powder, e.g. to be reconstituted with water, juice, milk, or any other suitable beverage prior to use. The term “beverage” or “drink” refers generally to any liquid or semi-solid form suitable for oral consumption by an individual that is now known or becomes known by those skilled in the art, and also includes concentrates, for example, frozen concentrates, and powders that can be dissolved in a suitable volume of liquid carrier to generate a so-called “instant” liquid or semi-solid for consumption by an individual. For example, when administered in the form of a beverage, the nutritional composition may be water-based, milk-based, tea-based, fruit juice-based, or some combination thereof.

Where the nutritional composition is mixed with, i.e., reconstituted with a liquid to form a beverage, the beverage composition will include between 20 and 60 g of the nutritional composition and between 150 ml and 400 ml of liquid to produce a beverage. In some instances, the composition is such that between 30 and 50 g, for example between about 30 g to about 40 g or about 35 g of the nutritional composition is combined with between about 200 ml and 300 ml, for example, about 250 ml of a liquid to produce a beverage. In other instances, the composition is such that between 40 g to about 45 g of the nutritional composition is combined with between about 200 ml and 300 ml, for example, about 250 ml of a liquid to produce a beverage.

In some instances, a beverage is provided that comprises, consists essentially of, or consists of about 80% to about 90% of a liquid with the remaining being the described nutritional composition.

In one embodiment, the nutritional composition consists essentially of, or consists of from about 75% to about 95% of a first protein source that includes from about 85% to about 99% of protein by weight of the first protein source and an endogenous source of tryptophan in an amount between about 1% to about 6% of tryptophan by weight of the first protein source; and from about 0.5% to about 4% of a second protein source consisting of an exogenous source of tryptophan such that the nutritional composition provides a ratio of tryptophan to LNAA in the range from about 0.080:1 to about 0.167:1 (which the skilled artisan will appreciate provides a ratio of large neutral amino acids to tryptophan in the range from about 6:1 to about 12.5:1).

In another embodiment the nutritional composition consists essentially of, or consists of (a) from about 75% to about 95% of a first protein source that includes from about 85% to about 99% of protein by weight of the first protein source and an endogenous source of tryptophan in an amount between about 1% to about 6% of tryptophan by weight of the first protein source; (b) from about 0.5% to about 4% of a second protein source consisting of an exogenous source of tryptophan such that the nutritional composition provides a ratio of tryptophan to large amino acids in the range from about 0.080:1 to about 0.167:1 and (c) a carbohydrate, an amino acid other than a LNAA, and a purine ribonucleoside monophosphate.

In another embodiment the nutritional composition consists essentially of, or consists of (a) from about 75% to about 95% of a first protein source that includes from about 85% to about 99% of protein by weight of the first protein source and an endogenous source of tryptophan in an amount between about 1% to about 6% of tryptophan by weight of the first protein source; (b) from about 0.5% to about 4% of a second protein source consisting of an exogenous source of tryptophan such that the nutritional composition provides a ratio of tryptophan to large neutral amino acids in the range from about 0.080:1 to about 0.167:1 and (c) a maltodextrin, theanine, and adenosine-5′-monophosphate (AMP).

Advantageously, the described nutritional composition is suitable for use in a method of supporting or enhancing at least one, and in some instances, two or more, qualitative and/or quantitative aspect(s) of sleep in a subject (e.g., a human). In one aspect, supporting or enhancing qualitative and/or quantitative aspect(s) of sleep comprises one or more of the following:
(i) promoting falling asleep;
(ii) inducing or supporting a healthy sleep pattern (i.e., sleep cycles including REM and non-REM sleep);
(iii) increasing total sleep time;
(iv) improving sleep efficiency;
(v) reducing or preventing sleep disturbances, i.e., sleeping more time while in bed;
(vi) increasing subjective and or objective measures of deep sleep;
(vii) feeling more refreshed at waking up;
(viii) feeling more energized and/or having a better mood during daytime.

Accordingly, a method for supporting or enhancing at least one, and in some instances, two or more, of the above qualitative and/or quantitative aspect(s) of sleep in a subject (e.g., a human) is contemplated. The method includes administering to the subject the nutritional compositions described above. Accordingly, it will be appreciated that the method may be effective for treating or mitigating insomnia or occasional sleeplessness, improving daytime energy and/or promoting nighttime relaxation. The methods comprise the administration of the unique nutritional compositions described above to reduce sleep onset latency and improve the quality of sleep by reducing the number of awakenings during sleep and/or increasing the duration of sleep, and or improving objective and subjective ratings of sleep quality. Without wishing to be bound by theory, it is also believed that the specific combination of described components of the nutritional compositions promotes regeneration during sleep (sleep recovery processes) and eventual negative effects of lack of sleep are reduced. As a result, the described nutritional composition and methods provide a safe and natural way of supporting or improving sleep quality without the use of hormones or pharmaceutical sedatives.

Measurements of the above qualitative and/or quantitative aspects may be made according to Halson et al., Optimisation and Validation of a Nutritional Intervention to Enhance Sleep Quality and Quantity, Nutrients 2020, 12, 2579. In that regard, the following methods may be used:

### Sleep Assessment

In one exemplary method, sleep may be recorded using polysomnography equipment with a standard montage of electrodes. Electrodes can be applied in the 60 min prior to lights out and include three electroencephalograms (C4-M1, F4-M1, O2-M1), two electrooculograms (left/right outer canthus), and a submental electromyogram. Stages of sleep may be identified as non-rapid eye movement sleep (stages N1, N2, N3) and rapid eye movement sleep (R), with N1 considered the lightest phase of sleep and N3 considered the deepest phase. The following dependent variables may be calculated from each sleep recording: total sleep time (min), the time spent in any stage of sleep (i.e., N1, N2, N3, R) during time in bed; time spent in stages N1, N2, N3 and R sleep (min); sleep onset latency (min), the time between lights-out to the first epoch of any stage of sleep (i.e., N1, N2, N3, R); wake after sleep onset (min), the time spent in bed awake minus sleep onset latency; sleep efficiency (%), total sleep time divided by time in bed×100; arousals, (count); arousals in NREM (count); arousals in REM (count); awakenings (count); stage shifts (count); stage R onset latency (min); and stage N3 onset latency.

Sleep assessment methods other than polysomnography may be used to measure sleep and/or quality of sleep (i.e., total sleep time and/or sleep efficiency) such as but not limited to questionnaires/diaries (e.g., Pittsburgh Sleep Quality Index (PSQI)), actigraph devices and consumer wearables (e.g., Biostrap, Wellue, Fitbit, Whoop, and Oura). Actigraphy generally refers to a validated method of objectively measuring sleep parameters and average motor activity over a period of days to weeks using a noninvasive accelerometer that typically housed in a small device that is worn like a wristwatch. Some of these actigraph devices are capable of tracking sleep duration and sleep stages.

Sleep assessment may include total sleep time (TST) and or sleep efficiency (SE). Total sleep time generally refers to the total amount of sleep time scored during the total recording time. This includes time from sleep onset to sleep offset and is distributed throughout the sleep time as minutes of Stage N1 sleep, Stage N2 sleep, Stage N3, and rapid eye movement (REM) sleep. Sleep efficiency (SE) generally refers to the ratio between the time a person spends asleep, and the total time dedicated to sleep (i.e., the total amount of time in bed). It is given as a percentage. SE of 80% or more is considered normal/healthy with most young healthy adults displaying SE above 90%.

### Subjective Sleepiness

Subjective sleepiness may be assessed using the Karolinska sleepiness scale (KSS). The KSS is a 9-point scale where 1=“extremely alert”, and 9=“very sleepy, great effort to keep awake, fighting sleep”.

### Subjective Sleep Quality, Sleep Duration, Sleep Latency

Sleep quality may be assessed using a 7-point scale, where 1=“extremely poor”, 2=“very poor”, 3=“poor”, 4=“average”, 5=“good”, 6=“very good”, and 7=“extremely good”. Subjects can be verbally asked “how much sleep do you think you got?” and “how long did it take you to fall asleep?”.

### Subjective Alertness and Self-Perceived Capacity

Subjective alertness may be assessed using a visual analogue scale. Subjects rate their current level of alertness by placing a vertical mark on a non-numeric, 100-mm line that was anchored with “not at all” at one end and “completely” at the other end. Self-perceived capacity can be assessed using two separate visual analogue scales. Subjects can rate their ability to perform as fast as possible by placing a vertical mark on a non-numeric, 100-mm line that was anchored with “not fast at all” at one end and “very fast” at the other end. Subject can rate their ability to perform as accurately as possible by placing a vertical mark on a non-numeric, 100-mm line that was anchored with “not accurately at all” at one end and “very accurately” at the other end.

### Cognitive Performance

Sustained attention can be assessed using the psychomotor vigilance task (PVT-192; Ambulatory Monitoring Inc., New York, N.Y., USA). The PVT is a hand-held device with an upper surface that contains a four-digit LED display and two push-button response keys. Subjects can attend to the LED display for the duration of the test (10 min) and can press the appropriate response key with the thumb of their dominant hand as quickly as possible after the appearance of a visual stimulus (presented at a variable interval of 2-10 s). If the correct response key was pressed, the LED display exhibited the subject's response time, in milliseconds, for 500 ms. If the wrong response key was pressed, an error message was displayed (ERR). If a response was made prior to the stimulus being presented, a false start message was displayed (FS). The dependent measures derived from the PVT included response time (ms); the number of lapses (i.e., response latency exceeding 500 ms) and the number of errors (i.e., false starts and incorrect button pushes). For all analyses, anticipated responses (i.e., those with response time less than 100 ms) were excluded. Cognitive throughput was assessed using a computer-based visual spatial-configuration search task. The task is self-paced and consists of 52 trials in which participants are required to search for a target (i.e., the number 5) amongst distractors (i.e., the number 2). Visual searches were performed for set sizes of 10, 20, 30, or 40 distractor stimuli. Set size was equally distributed across the task. The dependent variables obtained from the task were the number of errors and the time taken to complete the task.

The described forms of the composition are formulated for administration at various rates. Administration of one form prior to a desired period of sleep is contemplated. For example, for a substantial number of subjects (i.e., those who are awake during daytime hours) it is desirable to provide the nutritional composition for evening intake, e.g., by oral ingestion, of the described nutritional compositions. Of course, it will be understood that for those subjects who are awake during nighttime hours, it is desirable to provide for morning intake. In one embodiment, the nutritional composition is consumed for example, 120 minutes, 60 minutes or 30 minutes until immediately before going to bed, meaning an attempt to go to sleep. It is also contemplated that the described compositions are administered to subjects who are not taking any other sleep-improving agents.

While the concepts of the present disclosure are susceptible to various modifications and alternative forms, specific exemplary embodiments of the disclosure have been shown by way of example in the drawings. It should be understood, however, that there is no intent to limit the concepts of the present disclosure to the particular disclosed forms; the intention is to cover all modifications, equivalents, and alternatives falling within the spirit and scope of the invention as defined by the claims.

1. A nutritional composition comprising:
from about 75% to about 95% of a first protein source that includes from about 85% to about 99% of protein by weight of the first protein source, to provide an endogenous source of tryptophan in an amount between about 1% to about 6% of tryptophan, by weight of the first protein source; and
from about 0.5% to about 4% by weight of the nutritional composition of a second protein source consisting of an exogenous source of tryptophan;
wherein the nutritional composition provides a ratio of tryptophan to large neutral amino acids in the range from about 0.080:1 to about 0.167:1.

2. The nutritional composition according to claim 1 wherein the large neutral amino acid is selected from the group consisting of histidine, isoleucine, leucine, methionine, threonine, phenylalanine, valine, and tyrosine.

3. The nutritional composition according to claim 1, wherein the first protein source is selected from whey protein, whey protein concentrate, whey protein isolate, and mixtures thereof.

4. The nutritional composition according to claim 3, wherein the first protein source is whey protein isolate that comprises between about 85% to about 99% protein by weight of the whey protein isolate.

5. The nutritional composition according to claim 3, further comprising from about 3% to about 10% of a carbohydrate.

6. The nutritional composition according to claim 5, wherein the carbohydrate is a high glycemic index carbohydrate having a glycemic index in a range between about 85 and about 105.

7. The nutritional composition according to claim 6 wherein the high glycemic index carbohydrate comprises maltodextrin.

8. The nutritional composition according to claim 7 wherein the maltodextrin has a dextrose equivalent about 4 to about 20.

9. The nutritional composition according to claim 3 further comprising from about 0.25% to about 4% of theanine.

10. The nutritional composition according to claim 3 further comprising from about 0.00005% to about 0.0004% of adenosine-5′-monophosphate.

11. The nutritional composition according to claim 1 wherein the composition is a powder.

12. The nutritional composition according to claim 1 further comprising from about 150 ml to about 400 ml of a liquid.

13. The nutritional composition according to claim 12 wherein the liquid is water.

14. A nutritional composition comprising:
from about 75% to about 95% of a first protein source that includes from about 85% to about 99% of protein by weight of the first protein source, to provide an endogenous source of tryptophan in an amount between about 1% to about 6% of tryptophan, by weight of the first protein source;
from about 0.5% to about 4% by weight of the nutritional composition of a second protein source consisting of an exogenous source of tryptophan;
from 3% to about 10% of a carbohydrate;
from about 0.25% to about 4% of theanine; and,
from about 0.00005% to about 0.0004% of adenosine-5′-monophosphate;
wherein the nutritional composition provides a ratio of tryptophan to large neutral amino acids in the range from about 0.080:1 to about 0.167:1.

15. A method of supporting or enhancing at least one, preferably at least two or more, qualitative and/or quantitative aspect(s) of sleep in a subject, the aspects selected from (i) promoting falling asleep; (ii) inducing or supporting a healthy sleep pattern; (iii) increasing the total sleep time, (iv) improving sleep efficiency, (v) reducing or preventing sleep disturbances/sleeping more time while in bed; (vi) increasing subjective and or objective measures of deep sleep; (vii) feeling more refreshed at waking up; (viii) feeling more energized and/or having a better mood during daytime comprising administering the composition according to claim 1.

16. The method according to claim 15, wherein the subject is a human subject, preferably an adult subject, more preferably an adult subject not taking any other sleep-improving agents.

17. The method according to claim 15, wherein the composition is administered within 120 minutes, preferably within 90 minutes, prior to bedtime.

18. A method for reducing sleep onset latency comprising administering a beverage comprising:
from about 150 ml to about 400 ml of a liquid;
from about 85% to about 95% of a whey protein isolate that comprises between about 85% to about 99% protein by weight of the whey protein isolate and provides an endogenous source of tryptophan in an amount between about 1% to about 6% of tryptophan by weight of the whey protein isolate; and
an exogenous source of tryptophan in an amount between about 0.5% to about 2% such that the nutritional composition provides a ratio of tryptophan to large amino acids in the range from about 0.080:1 to about 0.167:1.

19. The method according to claim 18, wherein the large neutral amino acid is selected from the group consisting of histidine, isoleucine, leucine, methionine, threonine, phenylalanine, valine, and tyrosine.
